(19) United States
(12) Patent Application Publication
(10) Pub. No.: US 2022/0192754 A1
(43) Pub. Date: Jun. 23, 2022

(54) SYSTEM AND METHOD TO CHECK CUT PLANE ACCURACY AFTER BONE REMOVAL

(71) Applicant: THINK SURGICAL, INC., Fremont, CA (US)

(72) Inventors: Daniel Patrick Bonny, Fremont, CA (US); Yasmine Haddad, Fremont, CA (US); Timothy J. Pack, Fremont, CA (US); Eustache Felenc, Fremont, CA (US); Joel Zuhars, Fremont, CA (US)

(73) Assignee: THINK SURGICAL, INC., Fremont, CA (US)

(21) Appl. No.: 17/600,717

(22) PCT Filed: Apr. 13, 2020
(86) PCT No.: PCT/US2020/027885

§ 371 Date: Oct. 1, 2021

Related U.S. Application Data
(60) Provisional application No. 62/832,736, filed on Apr. 11, 2019.

(51) Int. Cl. A61B 34/20; A61B 17/15; A61B 17/17

(52) U.S. Cl. CPC A61B 34/20; A61B 2034/104; A61B 17/1764; A61B 17/155

(57) ABSTRACT

A device for checking post cut plane accuracy and alignment following bone removal in a bone of a patient during a computer-assisted surgical procedure to create a bone surface is provided. The device includes a body having an axis and adapted to contact the bone surface. One or more alignment features are associated with the body and are accessible when the body is in contact with the bone surface. Each of the one or more alignment features has a known orientation and position relative to the axis. A method for checking post cut plane accuracy and alignment following removal of bone from a patient to create a bone surface during a computer-assisted surgical procedure is also provided. A computer-assisted surgical system is provided that includes a tracking system, a tracked digitizer probe, the aforementioned device, a tracked surgical device, and one or more computers with software for determining the orientation.

### TECHNICAL FIELD

The present invention generally relates to computer assisted surgery, and more specifically to systems and methods for checking post cut plane accuracy and alignment following a bone cut or other removal during a surgical procedure.

### BACKGROUND

Computer-assisted orthopedic surgery is an expanding field having applications in total joint arthroplasty (TJA), bone fracture repair, maxillofacial reconstruction, and spinal reconstruction. For example, the TSOLUTION ONE® Surgical System (THINK Surgical, Inc., Fremont, Calif.) aids in the planning and execution of total hip arthroplasty (THA) and total knee arthroplasty (TKA). The TSOLUTION ONE® Surgical System includes: a pre-operative planning software program to generate a surgical plan using an image data set of the patient's bone and computer-aided design (CAD) files of several implants; and an autonomous surgical robot that precisely mills the bone to receive an implant according to the surgical plan. In order for the computer-assisted surgical system to accurately prepare a bone, the bone needs to be registered to the surgical system. The registration procedure maps the surgical plan onto the spatial position and orientation (POSE) of the bone in the coordinate system of the surgical system. Several registration procedures are known in the art, illustratively including pin-based, point-to-point matching, point-to-surface matching, laser scanning, and image-free registration as described in U.S. Pat. Nos. 5,951,475; 6,033,415; 8,287,522; and 8,010,177.

Total knee arthroplasty (TKA) is a surgical procedure in which the articulating surfaces of the knee joint are replaced with prosthetic components, or implants. TKA requires the removal of worn or damaged articular cartilage and bone on the distal femur and proximal tibia. The removed cartilage and bone is then replaced with synthetic implants, typically formed of metal or plastic, to create new joint surfaces. The position and orientation (POSE) of the removed bone, referred to as bone cuts or resected bone, determines the final placement of the implants within the joint. Generally, surgeons plan and create the bone cuts so the final placement of the implants restores the mechanical axis or kinematics of the patient's leg while preserving the balance of the surrounding knee ligaments. Even small implant alignment errors outside of clinically acceptable ranges correlate to significantly worse outcomes and increased rates of revision surgery. In TKA, creating the bone cuts to correctly align the implants is especially difficult because the femur requires at least five planar bone cuts to receive the femoral prosthesis.

FIG. 1 illustrates a three dimensional (3-D) model of a patient's distal femur 10 and a 3-D model of the femoral prosthesis 12 for a TKA procedure. The planar cuts on the distal femur that must be aligned in at least five degrees of freedom to ensure a proper orientation: anterior-posterior translation, proximal-distal translation, external-internal rotation, varus-valgus rotation, and flexion-extension rotation. Any misalignment or deviation in any one of the planar cuts or orientations may have drastic consequences on the final result of the procedure, including patient outcomes, implant wear, and the possibility for revision surgery. The final placement of the femoral prosthesis model 12 on the bone model 10 defines the bone cut planes (shaded regions of the bone model 10) where the bone is cut intra-operatively to receive the prosthesis as desired. In TKA, the planned cut planes generally include the anterior cut plane 14, anterior chamfer cut plane 16, the distal cut plane 18, the posterior chamfer cut plane 20, the posterior cut plane 22, and the tibial cut plane (not shown).

Guides, also referred to synonymously as cutting blocks, cutting jigs, alignment fixtures; are commonly used to aid in creating the bone cuts needed in orthopedic surgery. The guides include guide slots to restrict or align a bone removal device, such as an oscillating saw, in the correct bone resection plane. Guides are advantageous for several reasons. For one, the guide slots stabilize the bone removal device during cutting to ensure the bone removal device does not deflect from the desired plane due to the organic curvatures of the bone surface and as different density materials are engaged. Additionally, a single guide may contain multiple guide slots (referred to herein as an N-in-1 cutting block) which can define more than one cutting plane to be accurately resected, such as a 4-in-1 block, 5-in-1 block . . . N-in-1 block. Thus, the surgeon can quickly resect two or more planes once the cutting guide is accurately oriented on the bone. Still another advantage is that the guide slots and the working end of the oscillating saw are typically planar in shape and relatively thin, which make them ideal for creating planar bone cuts. The advantages of using a guide are apparent, however, for the guide to confer these advantages, the guides still needs to be accurately positioned on to the bone prior to executing the cut. In fact, the placement of the guide slots on the bone remains one of the most difficult, tedious, and exacting tasks for surgeons during TKA.

Various techniques have been developed to help a surgeon correctly align the guide slots on the bone. One system and method for aligning a cutting guide on the bone is described in U.S. Patent App. Pub. No. 2018/0344409 assigned to the assignee of the present application. With reference to FIG. 2A and FIG. 2B thereof, illustrate perspective views of a distal cutting guide 30. FIG. 2A is a front elevation view of the distal cutting guide 30 and FIG. 2B is a perspective view thereof. In general, cutting guides 30 and alignment guides used herein are made of a rigid or semi-rigid material, such as stainless steel, aluminum, titanium, polyetheretherketone (PEEK), polyphenylsulfone, acrylonitrile butadiene styrene (ABS), and the like. The distal cutting guide 30 includes a guide portion 32 and an attachment portion 34. The guide portion 32 includes a guide slot 36 and a bottom surface 40. The guide slot 36 is for guiding a surgical saw in creating the planned distal cut CP (see FIG. 2D) on the femur F. The bottom surface 40 may abut against one or more bone pins P that are placed on the femur F as shown in FIG. 2C. The attachment portion 34 and the guide portion 32 clamp to the bone pins P using fasteners 38. Here, a virtual pin plane PP for the distal cut guide 30 is defined in a surgical plan by planning software using the POSE of the planned distal cut plane CP (shown in FIG. 2D), and the distance between the guide slot 36 and the bottom surface 40 of the guide portion 32. The planning software may also use the known width of the bone pins P. For example, the pin plane PP may be defined by proximally translating the planned distal cut plane CP by the distance between the guide slot 36 and the bottom surface 40 of the distal cutting guide 30. The software may further proximally translate the planned distal cut plane CP by an additional half width of the pins P. Therefore, when the cutting guide 30 is clamped to the bone pins P as shown in FIG. 2D, the guide slot 36 is aligned with the planned distal cut plane CP.

Other methods have also been developed to alleviate the use of cutting guides. Haptic and semi-active robotic systems allow a surgeon to define virtual cutting boundaries on the bone. The surgeon then manually guides a cutting device while the robotic control mechanisms maintain the cutting device within the virtual boundaries. The cutting device may encounter curved surfaces on the bone causing the device to skip or otherwise deflect away from the resection plane. The resulting planar cuts would then be misaligned, or at least difficult to create since the cutting device cannot be oriented directly perpendicular to the curved surface of the bone to create the desired bone cut. Cutting guides on the other hand are removably fixed directly against the bone, and therefore deflection of the cutting device is greatly decreased. However, even when using a cut guide with a cutting device, illustratively including a surgical saw, various events such as saw blade skiving can lead to a cutting error.

In active robotic surgery, cuts are controlled by a cut file to autonomously create the bone cuts. A robot arm controlling an end-effector actively manipulates the end-effector to create the bone cuts according to the instructions in the cut-file. However, all active robotic systems are prone to some error due to imaging errors, segmentation errors, registration errors, bone movement, tracking errors, etc. which may have a small effect on the final POSE of the bone cuts.

Thus, there exists a need for a device and method to check or monitor the positional accuracy of the bone surfaces created by bone removal during a computer-assisted surgical procedure that are intended to have a specified orientation.

### SUMMARY OF THE INVENTION

A device for checking post cut plane accuracy and alignment following bone removal in a bone of a patient during a computer-assisted surgical procedure to create a bone surface is provided. The device includes a body having an axis and adapted to contact the bone surface. One or more alignment features are associated with the body and are accessible when the body is in contact with the bone surface. Each of the one or more alignment features has a known orientation and position relative to the axis.

A method for checking post cut plane accuracy and alignment following removal of bone from a patient to create a bone surface during a computer-assisted surgical procedure is also provided. The method includes tracking or fixing the bone relative to a computer-assisted surgical device. The patient bone is registered to pre-operative bone data and a device is attached to cut bone surface. A device is attached to the bone surface. A tracked object is assembled with a known axis with the one or more alignment features of the device. An orientation of the bone surface is determined if the orientation corresponds to a planned bone cut on the pre-operative bone data based on information from the tracked object.

A computer-assisted surgical system is provided that includes a tracking system, a tracked digitizer probe, the aforementioned device, a tracked surgical device, and one or more computers with software for determining if an orientation of the bone surface corresponds to a planned bone cut on the pre-operative bone data based on information from at least one of the digitizer probe or the tracked surgical device.

### BRIEF DESCRIPTION OF THE DRAWINGS

The present invention is further detailed with respect to the following drawings that are intended to show certain aspects of the present of invention, but should not be construed as limit on the practice of the invention, wherein:

FIG. 1 illustrates a prior art example of the planned cut planes on a three-dimensional model of a bone so as to receive a femoral knee implant;

FIG. 2A illustrates a front view of a prior art distal cutting guide in assembled form;

FIG. 2B illustrates a perspective view of the prior art distal cutting guide of FIG. 2A in exploded form;

FIG. 2C illustrates a prior art set of pins driven coincident with a virtual pin plane in a femoral bone;

FIG. 2D illustrates the prior art distal cutting guide of FIG. 2A assembled to the pins of FIG. 2C;

FIG. 3 depicts a method for checking post cut plane accuracy and alignment following a bone cut during a surgical procedure in accordance with embodiments of the invention;

FIG. 4 illustrates a partially transparent front view of an inventive device with channels shown as dotted lines in accordance with embodiments of the invention and a long axis defined by line A-A′;

FIGS. 5A-5C are a series of photographs of the post cut confirmation device of FIG. 4 positioned on a cut bone surface plane made in the femoral bone with a tracked object held above the channels in the medial-lateral direction (FIG. 5A), in the anterior-posterior direction (FIG. 5B), and at a 45 degree angle from the sagittal and/or coronal plane (FIG. 5C), respectively;

FIG. 6 is a photograph of a tracked digitizer probe held above the channel in the anterior-posterior direction showing the tracked distal end of the probe of FIG. 5B;

FIG. 7 depicts a front view of an inventive device with alignment markings in accordance with embodiments of the invention.

FIG. 8 depicts a surgical system in the context of an operating room (OR) with a hand-held surgical tool for implementing the method of FIG. 1 in accordance with embodiments of the invention; and

FIG. 9 depicts a surgical system in the context of an operating room (OR) with a surgical robot for implementing the method of FIG. 1 in accordance with embodiments of the invention.

### DETAILED DESCRIPTION

The present invention has utility as a system and method for checking post bone removal plane accuracy on a bone surface and alignment following a bone cut or other form of bone removal during a surgical procedure. The present invention will now be described with reference to the following embodiments. As is apparent by these descriptions, this invention can be embodied in different forms and should not be construed as limited to the embodiments set forth herein. Rather, these embodiments are provided so that this disclosure will be thorough and complete, and will fully convey the scope of the invention to those skilled in the art. For example, features illustrated with respect to one embodiment can be incorporated into other embodiments, and features illustrated with respect to a particular inventive embodiment may be deleted from the embodiment. In addition, numerous variations and additions to the embodiments suggested herein will be apparent to those skilled in the art in light of the instant disclosure, which do not depart from the instant invention. Hence, the following specification is intended to illustrate some particular embodiments of the invention, and not to exhaustively specify all permutations, combinations, and variations thereof.

Further, it should be appreciated that although the systems and methods described herein make reference to total knee arthroplasty, the systems and methods may be applied to other computer-assisted surgical procedures involving other bones and joints in the body to check cut plane accuracy illustratively including the hip, ankle, elbow, wrist, skull, and spine, as well as revision of initial repair or replacement of any of the aforementioned bones or joints.

All publications, patent applications, patents and other references mentioned herein are incorporated by reference in their entirety.

It is to be understood that in instances where a range of values are provided that the range is intended to encompass not only the end point values of the range but also intermediate values of the range as explicitly being included within the range and varying by the last significant figure of the range. By way of example, a recited range of from 1 to 4 is intended to include 1-2, 1-3, 2-4, 3-4, and 1-4.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

Unless indicated otherwise, explicitly or by context, the following terms are used herein as set forth below.

As used in the description of the invention and the appended claims, the singular forms “a,” “an” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

As used herein, the term “digitizer” refers to a device capable of measuring, collecting, recording, or designating the position of physical coordinates in three-dimensional space. For example, the ‘digitizer’ may be: a “mechanical digitizer” having passive links and joints, such as the high-resolution electro-mechanical sensor arm described in U.S. Pat. No. 6,033,415; a non-mechanically tracked digitizer probe (e.g., optically tracked, electromagnetically tracked, acoustically tracked, and equivalents thereof) as described for example in U.S. Pat. No. 7,043,961; or an end-effector of a robotic device.

As used herein, the term “digitizing” refers to the collecting, measuring, and/or recording the position of physical points in space with a digitizer.

As used herein, the term “pre-operative bone data” refers to bone data used to pre-operatively plan a procedure before making modifications to the actual bone. The pre-operative bone data may include one or more of the following. An image data set of a bone (e.g., computed tomography, magnetic resonance imaging, ultrasound, x-ray, laser scan), a virtual generic bone model, a physical bone model, a virtual patient-specific bone model generated from an image data set of a bone, or a set of data collected directly on a bone intra-operatively commonly used with imageless computer-assist devices.

Also described herein are “computer-assisted surgical systems.” A computer assisted surgical system refers to any system requiring a computer to aid in a surgical procedure. Examples of computer-assisted surgical systems include 1−N degree of freedom hand-held surgical systems, tracking systems, tracked passive instruments, active or semi-active hand-held surgical devices and systems, autonomous serial-chain manipulator systems, haptic serial chain manipulator systems, parallel robotic systems, or master-slave robotic systems, as described in U.S. Pat. Nos. 5,086,401, 7,206,626, 8,876,830, and 8,961,536, U.S. Pat. App. No. 2013/0060278, and PCT. Intl. App. No. US2016/051713. In particular inventive embodiments, the surgical system is a robotic surgical system as described below. In particular inventive embodiments, the surgical system is a 2-DOF articulating device as described in U.S. patent application Ser. No. 15/778,811. The surgical system may provide autonomous, semi-autonomous, or haptic control and any combinations thereof. In addition, a user may manually maneuver a tool attached to the surgical system while the system provides at least one of power, active, or haptic control to the tool.

As used herein, the term “registration” refers to the determination of the POSE and/or coordinate transformation between two or more objects or coordinate systems such as a computer-assist device, a bone, pre-operative bone data, surgical planning data (i.e., an implant model, cut-file, virtual boundaries, virtual planes, cutting parameters associated with or defined relative to the pre-operative bone data), and any external landmarks (e.g., a tracking marker array) associated with the bone, if such landmarks exist. Methods of registration known in the art are described in U.S. Pat. Nos. 6,033,415, 8,010,177, and 8,287,522.

Also, referenced herein is a surgical plan. For context, the surgical plan is created, either pre-operatively or intra-operatively, by a user using planning software. The planning software may be used to generate three-dimensional (3-D) models of the patient's bony anatomy from a computed tomography (CT), magnetic resonance imaging (MRI), x-ray, ultrasound image data set, or from a set of points collected on the bone intra-operatively. A set of 3-D computer aided design (CAD) models of the manufacturer's prosthesis are pre-loaded in the software that allows the user to place the components of a desired prosthesis to the 3-D model of the boney anatomy to designate the best fit, position, and orientation of the implant to the bone. The planning software may additionally or alternatively include tools to custom design an implant relative to a boney features.

As used herein, the term “real-time” refers to the processing of input data within milliseconds such that calculated values are available within 10 seconds of computational initiation.

Also used herein is the term “optical communication” which refers to wireless data transfer via infrared or visible light as described in U.S. patent application Ser. No. 15/505,167 assigned to the assignee of the present application and incorporated by reference herein in its entirety.

Also, as used herein, “bone cut” is defined to include various processes of bone removal intended to create a bone surface; besides mechanical sawing, techniques operative to form a bone cut are milling, drilling, chiseling, laser cutting, and water jet cutting.

Embodiments of the invention provide a post cut confirmation device for checking post cut plane accuracy and alignment following a bone cut or other form of bone removal that exposes a bone surface during a surgical procedure. Without use of embodiments of the inventive post cut confirmation device, potential bone cut errors may go undetected. During a robotic surgical procedure, pre-operative bone data (e.g., virtual bone model) with a known coordinate system is registered to the patient bone in the coordinate system of the robotic system. As the planned cut bone surfaces are known relative to the pre-operative bone data based on the pre-operative surgical plan, embodiments of the inventive post cut confirmation device are used to confirm the POSE of the cut bone surfaces by placing the inventive device against the bone surface to confirm whether the cut or other bone removed conforms to the planned cuts. The post cut confirmation device is readily made of a sterilizable plastic, composite materials, metals, stainless steel, other alloys, or a combination thereof. Not limiting illustrative examples of materials suitable for the post cut confirmation device include stainless steel, aluminum, titanium, carbide, polyetheretherketone (PEEK), polyphenylsulfone, acrylonitrile butadiene styrene (ABS), and the like. The post cut confirmation device is made of sterilizeable materials for reuse or may be disposed of following a single surgical procedure so as to preclude cross contamination even with intervening sterilization. A tracked object with a known axis (both position and orientation) is assembled with an alignment feature (e.g., a channel, a groove, a notch, a symbol, a marking) associated with the post cut confirmation device to confirm the POSE of the cut plane. The alignment feature is at a known POSE relative to an axis, A-A′ of the device that for example is parallel to a flat surface of the device. The axis A-A′ may be a long or short axis of the device. It is appreciated that a given alignment feature may be a channel, which is interior to the device or on a surface thereof. The measured axis of the inserted tracked object can be used to measure the angle and distance of the cut surface on the patient's bone. Examples of tracked objects illustratively include a tracked digitizer probe having an attached tracking array, or a mechanical digitizer arm having a digitizer probe as its distal end.

In a specific inventive embodiment, the post cut confirmation device is in the form of a drill guide that is attachable to the bone. The drill guide has one or more alignment features (e.g., channels or holes) to receive the digitizer probe. The drill guide may further include one or more guide holes to guide a surgical drill to create one or more holes in the bone to execute the surgical procedure. In another specific inventive embodiment, the post cut confirmation device is in the form of a saw guide that may be held manually or fixed against the cut bone. The saw guide has one or more alignment features (e.g., channels or holes) to receive the digitizer probe. The saw guide further includes a guide slot for guiding a surgical saw in creating one or more cut planes on the bone to execute the surgical procedure, such a distal-cut saw guide or a 4-in-1 cutting block. The alignment features in embodiments of the post cut confirmation device in some embodiments are oriented as follows: anterior-posterior for measuring both flexion-extension angle and proximal-distal distance; medial-lateral for measuring both varus-valgus angle and proximal-distal distance. In particular embodiments, the post cut confirmation device includes a single alignment feature oriented at 45 degrees from an anterior-posterior direction and/or medial-lateral direction of the confirmation device. (e.g., 45 degrees from the aforementioned anterior-posterior alignment feature or medial-lateral alignment feature). With a single 45-degree alignment feature, the flexion-extension angle is measured as follows: i) the tracked object is assembled with the 45-degree alignment feature; ii) the position and orientation of the tracked object is determined by the tracking system (e.g., optical or mechanical); and iii) projecting the determined orientation onto a virtual sagittal plane defined on the bone model during planning. The varus-valgus angle is measured as follows: i) the tracked object is assembled with the 45-degree alignment feature; ii) the position and orientation of the tracked object is determined by the tracking system (e.g., optical or mechanical); and iii) projecting the determined orientation onto a virtual coronal plane defined on the bone model during planning. Thus, a single 45-degree alignment feature may be used to measure the flexion-extension angle, varus-valgus angle, and proximal-distal translation all in one.

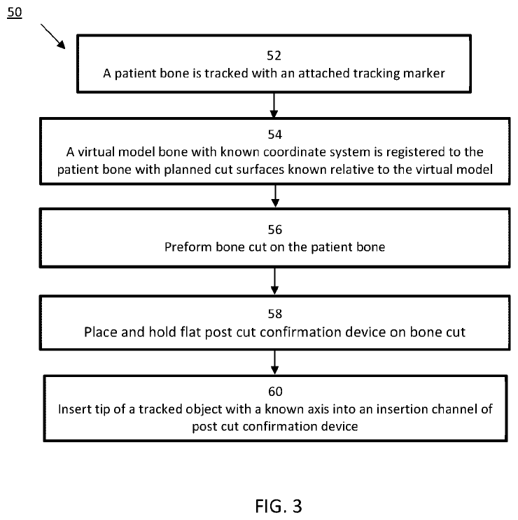

Referring now to the figures, FIG. 3 depicts an embodiment of a method 50 for checking post cut plane accuracy and alignment following a bone cut during a surgical procedure. A patient bone is tracked or fixed relative to a computer-assisted surgical system at Block 52. Pre-operative bone data is registered to the patient bone with planned cut surfaces known relative to the pre-operative bone data at Block 54. A bone cut is performed on the patient bone at Block 56. The post cut confirmation device is placed and held against the bone cut at Block 58. A tip of a tracked object with a known axis is assembled with an alignment feature of the post cut confirmation device at Block 60. A determination is made of the position and/or orientation of the bone cut on patient bone corresponds to the cut plan at Block 62. If the actual bone cut differs from the cut plan defined relative to the pre-operative bone data, a correction may be made to the actual bone cut if the difference is greater than a predetermined error margin at Block 64. If additional bone cuts are required at Block 66 the next bone cut is performed at Blocks 68, 56 and the confirmation steps performed in Blocks 58-66 are repeated on the subsequent bone cuts.

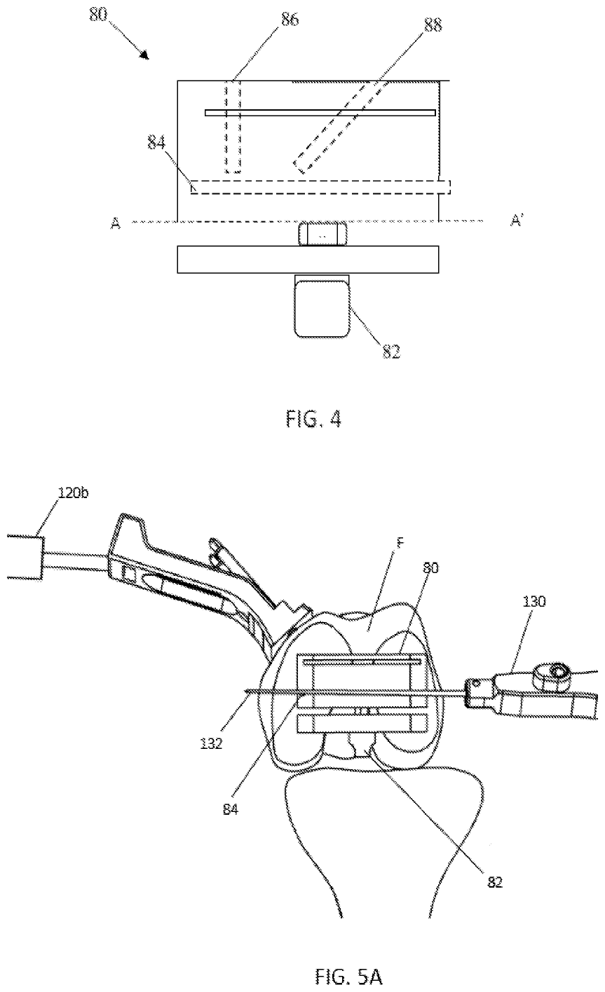

FIG. 4 is a side view of a particular embodiment of a post cut confirmation device 80, where the alignment features are channels (84, 86, 88) shown as dotted lines. The channels (84, 86, 88) are oriented in this embodiment in the anterior-posterior direction, in the medial-lateral direction, and at a 45 degree angle from the anterior-posterior or medial-lateral direction, respectively relative to the axis A-A′ that is shown along a flat lower surface of the device 80. Channel 84 is shown as a surface alignment feature where the tracked object can be placed on the channel 84, while channels 86 and 88 are interior to the volume of the device 80 where the tracked object can be placed into the channels 86 and 88. The channels (84, 86, 88) each independently have a diameter that is 1-5% larger than the diameter of the probe tip of the tracked object so as to provide a tight fit without play, thereby providing an accurate indication of the orientation of the bone cut with respect to the planned cut. Adjustment tab 82 which translates an adjustment plate 89 is present in some inventive embodiments to secure the position of the post cut confirmation device 80 on the bone, such as by way of clamping onto a securement feature on the bone. This securement feature illustratively includes bone pins, screws, a feature created on the bone (e.g., ridge or channel), or an additional alignment guide, drill guide, or saw guide.

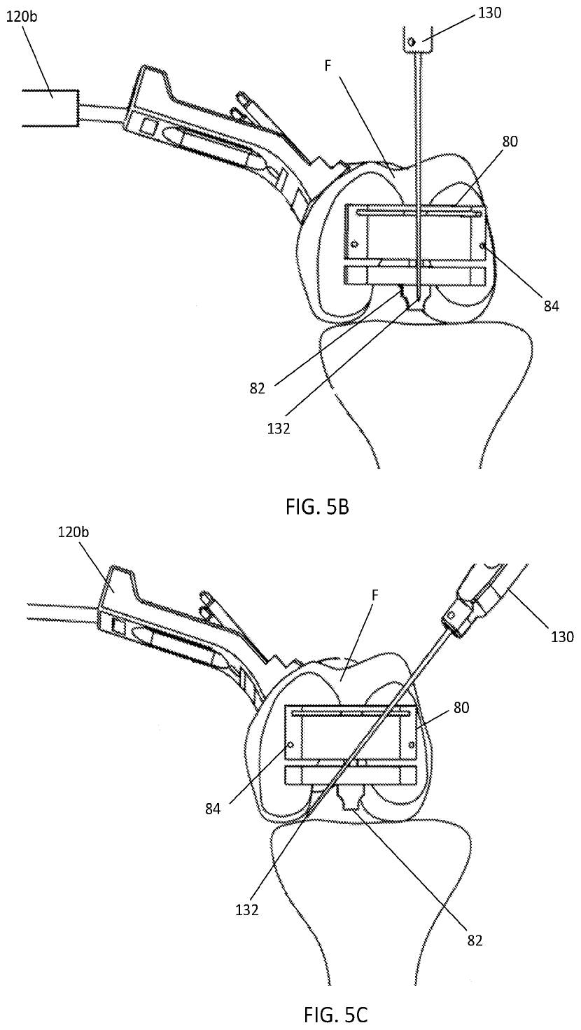

FIGS. 5A-5C are a series of images of the post cut confirmation device of FIG. 4 positioned on a cut plane made in the femoral bone F with a tracked object 130 held above the channels (84, 86, 88) in the medial-lateral direction, in the anterior-posterior direction, and at 45 degrees, respectively. Also visible in the images is a tracking marker array 120B that is used to track the position of the bone. During a measurement, the probe 132 is inserted into one or more of the channels (84, 86, 88). In FIG. 5A, the probe 132 is shown in position for obtaining a measurement in the medial-lateral direction by assembling the probe 132 onto the channel 84. In FIG. 5B the probe 132 is shown in position for obtaining a measurement in the anterior-posterior direction with channel 86. In FIG. 5C the probe 132 is shown in position for obtaining a measurement at 45 degrees with channel 88.

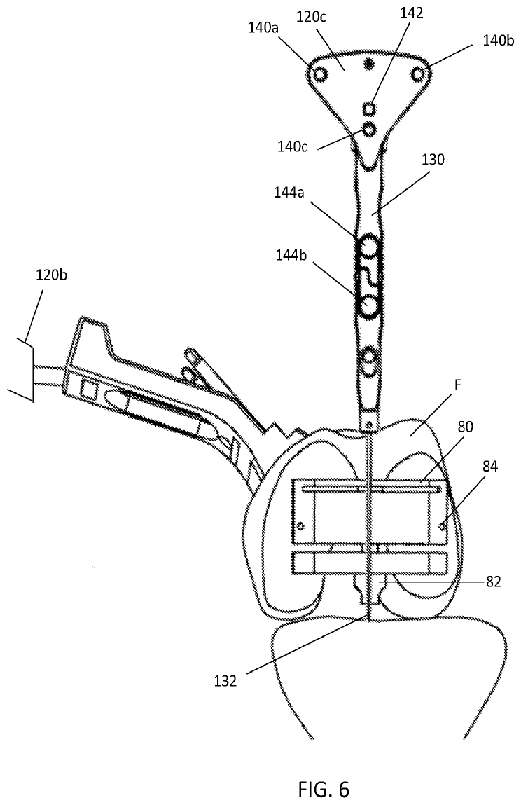

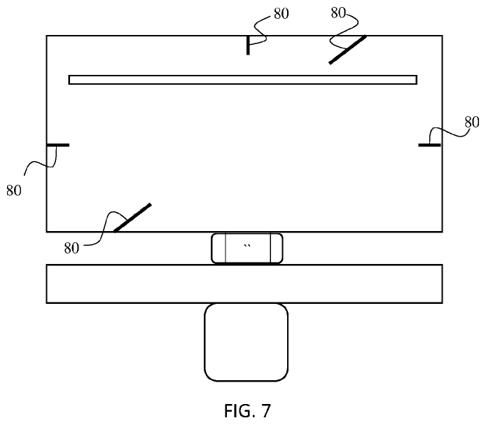

FIG. 6 is a photograph of a tracked digitizer probe 130 held above the channel 86 of the post cut confirmation device 80 in the anterior-posterior direction. A tracking array 120c is also visible and will be discussed in further detail with respect to the surgical system shown in FIG. 7.

FIG. 7 depicts an embodiment of a post cut confirmation device 80, where the alignment features are alignment markings (90a, 90b, 92a, 92b, 94a, 94b) on a top surface of the post cut confirmation device 80. Alignment markings 90a and 90b permit a user to align the tracked object in the medial-lateral direction, alignment markings 92a and 92b permit a user to align the tracked object in the anterior-posterior direction, and alignment marking 94a and 94b permit a user to align the tracked object at the aforementioned 45 degree angle. A user may assemble the tracked object with the post cut confirmation device 80 by resting the tracked object on the top surface of the confirmation device 80. Further, the distance between the top surface and the bottom surface that contacts the bone may be known to permit the surgical system to calculation the proximal-distal distance.

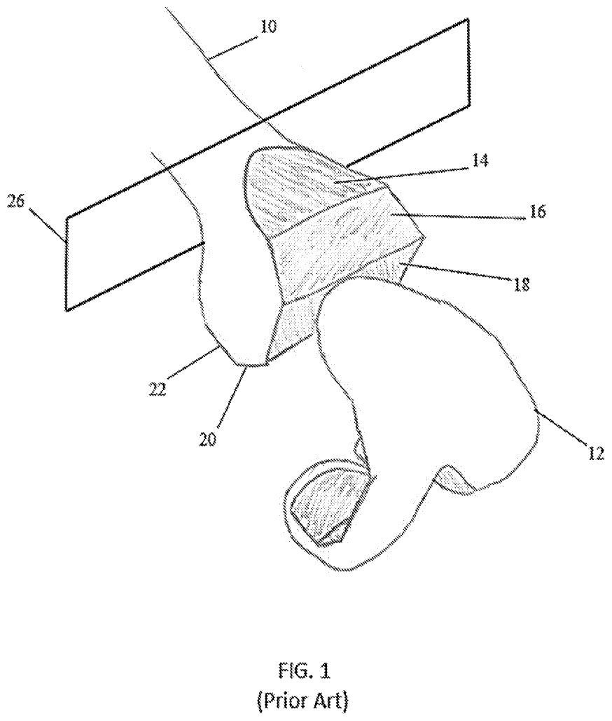

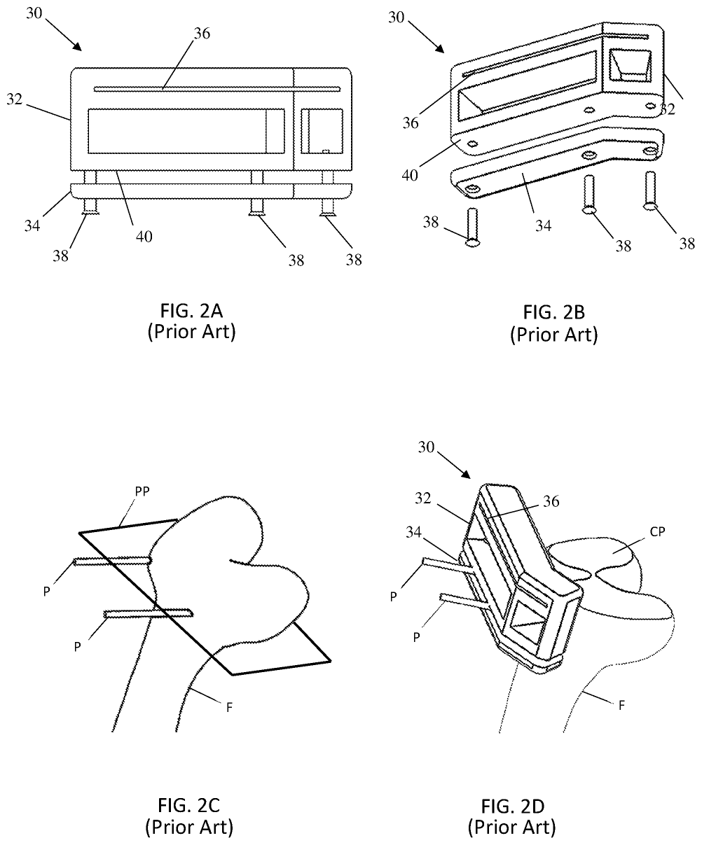

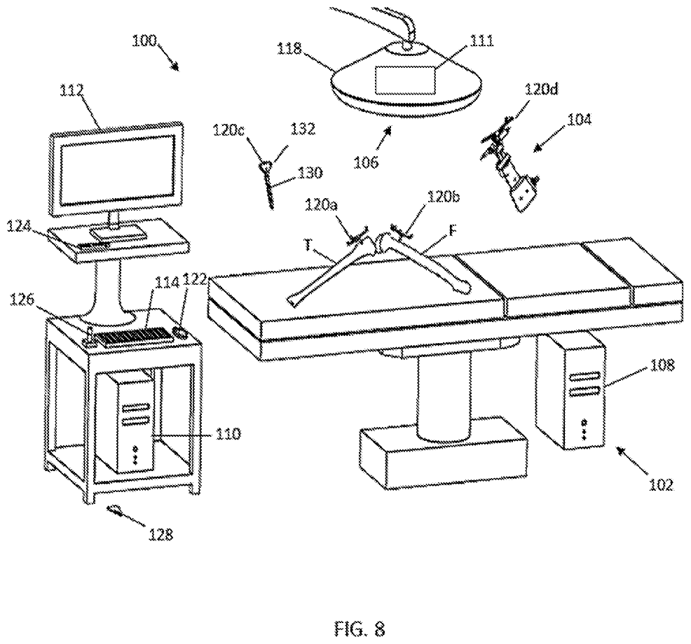

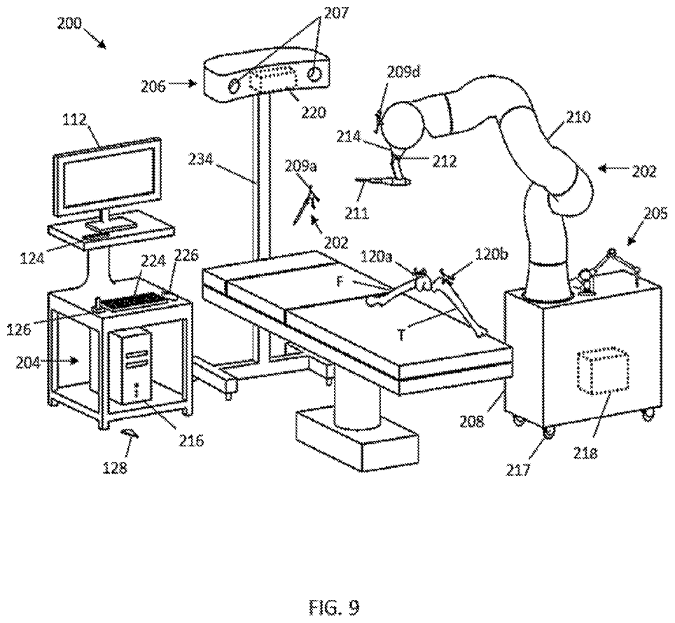

FIG. 8 depicts a surgical system 100 in the context of an operating room (OR) with a hand-held surgical tool 104 for implementing embodiment of the inventive method of FIG. 1. FIG. 9 depicts a surgical system 200 in the context of an operating room (OR) with a surgical robot 202 for implementing the embodiments of the method of FIG. 1. The systems shown in FIGS. 8 and 9 will be described in a single discussion with common elements having the same reference number.

The surgical system 100 of FIG. 8 is described in more detail in U.S. patent Ser. No. 15/778,811 assigned to the assignee of the present application. The 2-DOF surgical system 200 generally includes a computing system 102, a hand-held articulating surgical device 104 with a tracking array 120d, and a tracking system 106. The surgical system 100 is able to guide and assist a user in accurately placing pins coincident with a target pin plane that is defined relative to a subject's bone. The target plane is defined in a surgical plan and the pins permit the assembly of various cut guides and accessories to aid the surgeon in making the cuts on the femur and tibia to receive a prosthetic implant in a planned position and orientation.

The computing system 102 in some inventive embodiments includes: a device computer 108 including a processor; a planning computer 110 including a processor; a tracking computer 111 including a processor, and peripheral devices. Processors operate in the computing system 102 to perform computations associated with the inventive system and method. It is appreciated that processor functions are shared between computers, a remote server, a cloud computing facility, or combinations thereof.

In particular inventive embodiments, the device computer 108 may include one or more processors, controllers, software, data, utilities, and any additional data storage medium such as RAM, ROM or other non-volatile or volatile memory to perform functions related to controlling a surgical workflow and provide guidance to the user, controlling the actuation of the surgical device 104, controlling power to the surgical device (e.g., power to the drill), interpret pre-operative planning surgical data, and processing tracking or POSE data. In some embodiments, the device computer 108 is in direct communication with the optical tracking system 106 such that the optical tracking system 106 may identify trackable devices in the field of view (FOV), and the device computer 108 can control the workflow accordingly based on the identity of the tracked device. However, it should be appreciated that the device computer 108 and the tracking computer 111 may be separate entities as shown, or it is contemplated that their operations may be executed on just one or two computers depending on the configuration of the surgical system 100. For example, the tracking computer 111 may have operational data to directly control the workflow without the need for a device computer 108. Or, the device computer 108 may include operational data to directly read data detected from the optical cameras without the need for a tracking computer 111. In particular inventive embodiments, the device computer 108 is located on-board the surgical device 104 (e.g., in the hand-held portion of the surgical device 104). In any case, the peripheral devices allow a user to interface with the surgical system 100 and may include: one or more user interfaces, such as a display or monitor 112; and various user input mechanisms, illustratively including a keyboard 114, mouse 122, pendent 124, joystick 126, foot pedal 128, or the monitor 112 may have touchscreen capabilities.

The planning computer 110 is preferably dedicated to planning the procedure either pre-operatively or intra-operatively. For example, the planning computer 110 may contain hardware (e.g. processors, controllers, and memory), software, data, and utilities capable of receiving and reading medical imaging data, segmenting imaging data, constructing and manipulating three-dimensional (3D) virtual models, storing and providing computer-aided design (CAD) files, planning the POSE of the implants relative to the bone, generating the surgical plan data for use with the system 100, and providing other various functions to aid a user in planning the surgical procedure. The planning computer also contains software dedicated to defining target planes defined relative to the planned cut planes. The final surgical plan data may include an image data set of the bone, bone registration data, subject identification information, the POSE of the implants relative to the bone, the POSE of one or more target planes defined relative to the bone, and any tissue modification instructions. The final surgical plan is readily transferred to the navigation computer 108 and/or tracking computer 111 through a wired or wireless connection in the operating room (OR); or transferred via a non-transient data storage medium (e.g. a compact disc (CD), a portable universal serial bus (USB drive)) if the planning computer 110 is located outside the OR. The registered surgical planning data may then be transmitted to the surgical device 104. In particular embodiments, data is transferred from the planning computer 110, tracking computer 111, device computer 108, and any combination thereof by way of optical light as described in U.S. Pat. Pub. No. 20170245945 assigned to the assignee of the present application and incorporated by reference herein.

In a particular embodiment, the tracking system 106 is an optical tracking system as described in U.S. Pat. No. 6,061,644, having two or more optical camera (not shown because the cameras are situated inside a surgical lamp 118 and directed towards the surgical site) to detect the position of fiducial markers arranged on rigid bodies (tracking arrays) or integrated directly into the tracked devices. Illustrative examples of the fiducial markers include: an active transmitter, such as an LED or electromagnetic radiation emitter; a passive reflector, such as a plastic sphere with a retro-reflective film; or a distinct pattern or sequence of shapes, lines or other characters. A set of fiducial markers arranged on a rigid body is referred to herein as a tracking marker array (120a, 120b, 120c, 120d), however, the fiducial markers may be integrated and arranged directly onto the tracked devices. Each fiducial marker array (120a, 120b, 120c, 120d) or set of fiducial markers on each tracked device has a unique geometry/arrangement of fiducial markers, or a unique transmitting wavelength/frequency if the markers are active LEDS, such that the tracking system 106 can distinguish between each of the tracked objects and therefore act as the reference members associated with each tracked device.

In specific inventive embodiments, the tracking system 106 is built into a surgical lamp 118, which therefore limits the FOV of the optical cameras. However, in other embodiments the tracking system 106 and cameras are located on a boom, stand, or built into the walls or ceilings of the operating room. The tracking system computer 111 includes tracking hardware, software, data, and utilities to determine the POSE of objects (e.g., bones such as the femur F and tibia T, the surgical device 104) in a local or global coordinate frame. The POSE of the objects is referred to herein as POSE data, where this POSE data is readily communicated to the navigation computer 108.

The surgical system 100 further includes a tracked digitizer probe 130 as mentioned above and shown in greater detail in FIG. 6 for registering one or more bones and for use with inventive embodiments of the post cut confirmation device 80. With reference to FIG. 6, a detailed view of the tracked digitizer probe 130 is shown. The tracked digitizer probe 130 includes three or more fiducial markers (140a, 140b, 140c), an optical communications LED 142, two or more selection buttons (144a, 144b), and a probe tip 132. The fiducial marker arrays (140a, 140b, 140c) may be present on a tracking array 120c, or the fiducial markers (140a 140b, 140c) may be directly incorporated onto the probe 130 in a unique fashion to permit the tracking system 106 to identify the tracked digitizer probe 130. The optical communications LED 142 allows the tracked digitizer probe 130 to communicate with the tracking system 106 and/or device computer 108. The two or more selection buttons (144a, 144b) allows the user to select between the femur and tibia in a registration mode menu of a graphical user interface (GUI). The buttons (144a, 144b) also allows the user to click and collect a point during the registration procedure.

Referring now to surgical system 200 of FIG. 9, the surgical robot 302 may include a movable base 208, a manipulator arm 210 connected to the base 208, an end-effector 211 located at a distal end 212 of the manipulator arm 210, and a force sensor 214 positioned proximal to the end-effector 211 for sensing forces experienced on the end-effector 211. The base 208 includes a set of wheels 217 to maneuver the base 208, which may be fixed into position using a braking mechanism such as a hydraulic brake. The base 208 may further include an actuator to adjust the height of the manipulator arm 210. The manipulator arm 210 includes various joints and links to manipulate the end-effector 211 in various degrees of freedom. The joints are illustratively prismatic, revolute, spherical, or a combination thereof.

The computing system 204 generally includes a planning computer 216; a device computer 218; a tracking computer 220; and peripheral devices. The planning computer 216, device computer 218, and tracking computer 220 may be separate entities, one-in-the-same, or combinations thereof depending on the surgical system. Further, in some embodiments, a combination of the planning computer 216, the device computer 218, and/or tracking computer 220 are connected via a wired or wireless communication. The peripheral devices allow a user to interface with the surgical system components and may include: one or more user-interfaces, such as a display or monitor 122 for the graphical user interface (GUI); and user-input mechanisms, such as a keyboard 124, mouse or joystick 126, pendant 128, foot pedal 132, or the monitor 122 that in some inventive embodiments has touchscreen capabilities.

The planning computer 116 contains hardware (e.g., processors, controllers, and/or memory), software, data and utilities that are in some inventive embodiments dedicated to the planning of a surgical procedure, either pre-operatively or intra-operatively. This may include reading medical imaging data, segmenting imaging data, constructing three-dimensional (3D) virtual models, storing computer-aided design (CAD) files, providing various functions or widgets to aid a user in planning the surgical procedure, and generating surgical plan data. The final surgical plan may include pre-operative bone data, patient data, registration data including the POSE of a set of points P defined relative to the pre-operative bone data, implant position data, trajectory parameters, and/or operational data. The operational data may be a set of instructions for modifying a volume of tissue that is defined relative to the anatomy, such as a set of cutting parameters (e.g., cut paths, velocities) in a cut-file to autonomously modify the volume of bone, a set of virtual boundaries defined to haptically constrain a tool within the defined boundaries to modify the bone, a set of planes or drill holes to drill pins in the bone, a graphically navigated set of instructions for modifying the tissue, and the trajectory parameters for robotic insertion of an implant. The operational data specifically includes a cut-file for execution by a surgical robot to autonomously modify the volume of bone, which is advantageous from an accuracy and usability perspective. The surgical plan data generated from the planning computer 216 may be transferred to the device computer 218 and/or tracking computer 220 through a wired or wireless connection in the operating room (OR); or transferred via a non-transient data storage medium (e.g., a compact disc (CD), a portable universal serial bus (USB) drive) if the planning computer 216 is located outside the OR.

The device computer 218 in some inventive embodiments is housed in the moveable base 208 and contains hardware, software, data and utilities that are preferably dedicated to the operation of the surgical robotic device 202. This may include surgical device control, robotic manipulator control, the processing of kinematic and inverse kinematic data, the execution of registration algorithms, the execution of calibration routines, the execution of operational data (e.g., cut-files, the trajectory parameters), coordinate transformation processing, providing workflow instructions to a user, and utilizing position and orientation (POSE) data from the tracking system 206. In some embodiments, the surgical system 200 includes a mechanical digitizer arm 205 attached to the base 208. The digitizer arm 205 may have its own tracking computer connected with the device computer 218, or the device computer 218 may process the data of the digitizer arm 205 directly. The mechanical digitizer arm 205 may act as a digitizer probe akin to probe 130 that is assembled to a distal end of the mechanical digitizer arm 205 and may be inserted into the post cut confirmation device 80. In other inventive embodiments, the system includes a hand-held digitizer device 202 with a probe tip that may be inserted into the post cut confirmation device 80 and afford the function of the probe 130.

The tracking system 206 may be an optical tracking system that includes two or more optical receivers 207 to detect the position of fiducial markers (e.g., retroreflective spheres, active light emitting diodes (LEDs)) uniquely arranged on rigid bodies. The fiducial markers arranged on a rigid body are collectively referred to as a fiducial marker array (209a, 120a, 120b, 209d), where each fiducial marker array has a unique arrangement of fiducial markers, or a unique transmitting wavelength/frequency if the markers are active LEDs. An example of an optical tracking system is described in U.S. Pat. No. 6,061,644. The tracking system 206 may be built into a surgical light, located on a boom, a stand 234, or built into the walls or ceilings of the OR. The tracking system computer 220 may include tracking hardware, software, data, and utilities to determine the POSE of objects (e.g., bones B, surgical device 202) in a local or global coordinate frame. The POSE of the objects is collectively referred to herein as POSE data, where this POSE data may be communicated to the device computer 218 through a wired or wireless connection. Alternatively, the device computer 218 may determine the POSE data using the position of the fiducial markers detected from the optical receivers 207 directly.

The POSE data is determined using the position data detected from the optical receivers 207 and operations/processes such as image processing, image filtering, triangulation algorithms, geometric relationship processing, registration algorithms, calibration algorithms, and coordinate transformation processing.

The POSE data is used by the computing system 304 during the procedure to update the POSE and/or coordinate transforms of the bone B, the surgical plan, and the surgical robot 202 as the manipulator arm 210 and/or bone(s) (F, T) move during the procedure, such that the surgical robot 202 can accurately execute the surgical plan.

In another inventive embodiment, the surgical system 200 does not include an optical tracking system, but instead employs a mechanical arm 205 that may act as a tracking system 206 as well as a digitizer. If the bone is not tracked, a bone fixation and monitoring system may fix the bone directly to the surgical robot 202 to monitor bone movement as described in U.S. Pat. No. 5,086,401. In addition, bone motion may be detected with the use of the post cut confirmation device 80 as determined by any errors identified from the measured POSE of the cut surfaces relative to the planned cut surfaces.

### Other Embodiments

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the described embodiments in any way. Rather, the foregoing detailed description will provide those skilled in the art with a convenient roadmap for implementing the exemplary embodiment or exemplary embodiments. It should be understood that various changes may be made in the function and arrangement of elements without departing from the scope as set forth in the appended claims and the legal equivalents thereof.

1. A device for checking accuracy or alignment of a bone surface, the bone surface created on a bone during a surgical procedure, said device comprising:
a body having an axis and adapted to contact the bone surface; and
one or more alignment features associated with said body, said one or more alignment features accessible by a tracked object when said body is in contact with the bone surface, and each of said one or more alignment features having a known orientation and position relative to the axis.

2. The device of claim 1 wherein said device is made of one of plastic, composite materials, metals, stainless steel, or other alloys

3. The device of claim 1 wherein said device is made of stainless steel, aluminum, titanium, carbide, polyetheretherketone (PEEK), polyphenylsulfone, or acrylonitrile butadiene styrene (ABS).

4. The device of claim 1 wherein said one or more alignment features are oriented in one or more of the following directions: anterior-posterior, medial-lateral, and 45 degrees from the anterior-posterior or medial-lateral directions.

5. The device of claim 1 wherein said one or more alignment features are at least one of a channel, a groove, a notch, a symbol, or a marking.

6. The device of claim 1 wherein said one or more alignment features are one or more channels each independently having a diameter that is 1-5% larger than a diameter of a probe tip of the tracked object.

7. The device of claim 1 wherein the tracked object is a tracked digitizer probe.

8. The device of claim 1 wherein said device is in the form of a drill guide or a saw guide.

9. The device of claim 1 wherein said device is adapted to be fixed or held manually against the bone surface.

10. The device of claim 1 further comprising an adjustment mechanism and an adjustment plate, where the adjustment mechanism translates the adjustment plate to permit the device to clamp onto a securement feature on the bone.

11. The device of claim 1 wherein at least one of said one or more alignment features is parallel to a flat bottom surface of said body defining the axis.

12. The device of claim 1 wherein said one or more alignment features are each independently located on a surface of said body or within a volume of said body.

13. A method for checking accuracy or alignment of a bone surface, the bone surface created on a bone during a surgical procedure, said method comprising:
placing said device of claim 1 on the bone surface;
assembling a tracked object with a known axis with the one or more alignment features of said device; and
determining if an orientation of the bone surface corresponds to a planned bone surface based on information from the tracked object.

14. The method of claim 13 further comprising:
removing additional bone or reorienting the bone if the bone surface differs from the planned bone surface by an amount greater than a predetermined error margin.

15. The method of claim 13 wherein the bone is a femur.

16. The method of claim 13 wherein the tracked object is a tracked digitizer probe.

17. The method of claim 13 wherein measurements are taken in one or more of the following directions: anterior-posterior, medial-lateral, and at a 45 degree angle from the anterior-posterior direction or medial-lateral direction.

18. The method of claim 13 wherein the planned bone surface is defined relative to pre-operative bone data.

19. A computer-assisted surgical system, comprising:
a tracking system;
a tracked object; and
the device of claim 1.

20. The method of claim 18 wherein the pre-operative bone data is a virtual bone model and the planned bone surface is defined relative to the virtual bone model.
